(19) United States
(12) Patent Application Publication
(10) Pub. No.: US 2008/0085563 A1
(43) Pub. Date: Apr. 10, 2008

(54) Detection of Gadolinium Chelates

(75) Inventor: Ralph Magnotti, Westbrook, ME (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: IDEXX LABORATORIES, INC., Westbrook, ME (US)

(21) Appl. No.: 11/773,574

(22) Filed: Jul. 5, 2007

Related U.S. Application Data
(63) Continuation-in-part of application No. 11/545,430, filed on Oct. 10, 2006.

(51) Int. Cl. G01N 33/20

(52) U.S. Cl. 436/82

(57) ABSTRACT

A method for determining the presence or amount of a gadolinium chelate in a biological sample. The method includes contacting a biological sample with a dye selected from arsenazo III or chlorophosphonazo at low pH, and measuring the absorbance of the sample, thereby determining the presence or amount of gadolinium in the sample. A method for determining glomerular filtration (GFR) rate in a mammal. The method includes administering to the mammal an amount of a gadolinium chelate and determining the concentration levels of the chelate in biological samples taken from the animal at plurality of intervals following administration of the chelate. The concentration levels of the chelate are correlated to GFR.

### CROSS REFERENCE TO RELATED APPLICATIONS

This application is a continuation-in-part under 35 U.S.C. § 120 of U.S. patent application Ser. No. 11/545,430, filed Oct. 10, 2006 and entitled “Detection of Gadolinium Chelates,” which is hereby incorporated herein by reference.

### BACKGROUND OF THE INVENTION

1. Field of the Invention

The invention is related to the detection of gadolinium chelates in biological samples. In addition, the invention is related to the measurement of glomerular filtration rate (GFR) in animals to assess renal function in animals.

2. Description of Related Art

GFR (glomerular filtration rate) is established as a key indicator of kidney function. Unfortunately its utility for the diagnosis and management of kidney disease has not been fully realized, due in large part to the lack of an easily available, accurate method for its determination. Currently GFR in clinical practice is usually not determined directly. Instead, it is determined as an estimate (eGFR) calculated from measurement of serum creatinine. Unlike current methods for GFR, serum creatinine is easily measurable using commercial automated analyzers commonplace in hospital laboratories. However, despite considerable refinement over the years, creatinine-based eGFR has a number of drawbacks relative to the use of an authentic GFR. These include: insensitivity for the detection of the early stages of renal dysfunction when elevation of creatinine is small relative to its normal reference range, and imprecisions and inaccuracies which vary depending on the method used. In addition, the physiological variability of serum creatinine limits the diagnostic specificity of creatinine measurements. Because renal disease is often progressive, it is desirable to identify and treat it before renal failure ensues.

Plasma inulin clearance has long been accepted as a definitive method for measurement of GFR, although its application is costly, inconvenient and not widely available. GFR is calculated by measuring the rate of disappearance of inulin from the vascular circulation by analysis of its plasma concentration as a function of time following a single IV injection of the compound. Because inulin is eliminated from the body solely by glomerular filtration, and since it is not substantially bound to plasma components, its rate of clearance from plasma can be used to measure GFR. This method for GFR estimation has been evaluated in healthy dogs as well as dogs with reduced renal function.

In addition to inulin, other substances have long been established for measurement of GFR in humans and animals, including ^{99m}Tc-DTPA, ⁵¹Cr-EDTA and iohexyl. In addition, GFR has been estimated by nuclear or magnetic (MRI) imaging of the kidney after IV injection of a radiolabeled or paramagnetic substance. Unfortunately, these techniques require use of radioisotopes and specialized equipment not generally available to many practitioners.

Gadolinium-DTPA (Gd-DTPA; gadopentetate dimeglumine; MAGNEVIST®; Berlex Laboratories) has been validated against ^{99m}Tc-DTPA as a safe, non-radioactive indicator of GFR. Gd-DTPA has been proven to be safe even when used in patients with severe renal impairment. Gd-DTPA is routinely administered intravenously as a contrast agent in magnetic resonance imaging (MRI) examinations. A number of other gadolinium-chelate contrast agents are available commercially in the US: gadodiamide (OMNISCAN™; Amersham Health), gadoversetamide (OPTIMARK®; Mallinckrodt Medical), and gadoteridol (Prohance; Bracco). These agents exhibit renal clearance rates similar to Gd-DTPA and therefore may also be useful for measurement of GFR.

Widespread use of gadolinium chelates in such studies has been hindered, however, because the quantification of the chelates has required the separation of the chelates from interfering substances in the sample. Chromatographic separation and detection of gadolinium has been accomplished by HPLC methods, e.g., ion-pair chromatography in reverse-phase mode with on-line UV and radioactivity detection, reverse-phase high performance liquid chromatography (HPLC) with fluorescence detection and reverse-phase anion-exchange HPLC with UV detection. A major disadvantage of these methods is the requirement for dedicated high-complexity instrumentation, increasing both cost and inconvenience. Gadolinium can also be determined directly using neutron activation and magnetic resonance, but the instruments required for these techniques are costly and not widely available. As a consequence none of these methods has been adapted for use with the analyzers commonly used by hospital clinical chemistry services and performance of the GFR test has been restricted to a few specialized laboratories.

Accordingly, the inventors have recognized a need in the art for a sensitive, simple and reliable method for detecting gadolinium chelates in biological samples with clinical usefulness for evaluation of renal function.

### SUMMARY OF THE INVENTION

In one aspect, the invention is directed to a method for determining the presence or amount of a gadolinium chelate in a biological sample. The method includes contacting a biological sample with a dye selected from arsenazo III or chlorophosphonazo at a low pH and measuring the absorbance of the sample, thereby determining the presence or amount of gadolinium in the sample.

Another embodiment of this method involves contacting a biological sample with a reagent including arsenazo III at a pH of about 2.0 to about 4.0, or chlorophosphonazo at a pH of about 1.0 to about 3.0, and measuring the absorbance of the sample. The reagent may include HDMP (3-hydroxy-1,2-dimethyl-4(1H)-pyridone; CAS 30652-11-0; Deferiprone; FERRIPROX™), and/or a buffer to maintain the pH of the reagent between about 1.0 to about 4.0, depending upon the dye. The reagent may include a C₄-C₈ alkylsulfonate.

In another aspect, the invention is directed to a method for determining glomerular filtration (GFR) rate in a mammal. The method includes administering to the mammal an amount of a gadolinium chelate and determining the concentration level of the chelate in biological samples taken from the animal at a defined interval or plurality of timepoints following administration of the chelate. The determination may be accomplished by contacting the biological samples with arsenazo III at a pH of about 2.0 to about 4.0, or chlorophosphonazo at a pH of about 1.0 to about 3.0, and measuring the absorbance of the sample. The concentration levels of the chelate can be correlated to GFR.

In yet another aspect, the invention includes a colorimetric method for measuring glomerular filtration rate in an animal. This method includes administering to the animal a gadolinium chelate, collecting plasma or serum samples from the animal at various times following the administration, and determining the level of gadolinium in the samples. The determination may be accomplished by contacting the samples with a reagent including arsenazo III at a pH of about 2.0 to about 4.0, or chlorophosphonazo at a pH of about 1.0 to about 3.0, and measuring the absorbance of the samples. The absorbances of the samples are compared to the amount of time following the administration that they were collected, thereby determining the glomerular filtration rate.

Other aspects of the method of the invention include the absence of HPLC for biological samples. In addition, HDMP may be added to the reagent containing the dye.

### BRIEF DESCRIPTION OF THE FIGURES

FIG. 1 is a graph showing the results of an experiment to measure a gadolinium chelate in water at low pH.

FIG. 2 is a graph showing the results of an experiment to measure a gadolinium chelate in cat serum.

FIG. 3 is a graph showing the results of an experiment to measure a gadolinium chelate in canine serum with the removal of interfering cations using HDMP.

FIG. 4 is a graph showing the results of an experiment using the gadolinium-DTPA and arsenazo III at varying pH.

FIG. 5 is a graph showing the results of an experiment using the method of the invention for three types of gadolinium-DTPA and bovine fluoride-oxalate plasma (BF-OP).

FIG. 6 is a graph showing the results of an experiment using the method of the invention for three commercially-available DTPA chelates.

FIG. 7 is a graph showing shows the absorption spectra of chlorophosphonazo and two solutions containing chlorophosphonazo and varying concentrations of a gadolinium chelate.

FIG. 8 is a graph showing the results of an experiment using the method of the invention for bovine plasma using various concentrations of gadolinium-DPTA (MAGNEVIST®).

FIG. 9 is a graph showing the results of an experiment using the method of the invention to show the comparison of the gadolinium concentrations measured in the serum by a method of the invention and the gadolinium concentrations measured in the plasma by ICP-MS.

FIG. 10 shows the logarithmic plot of gadolinium concentration against time for ICP and arsenazo III-based method of the invention. GFR can be calculated as the slope of the regression line multiplied by the volume distribution (obtained from the line intercept and dose).

FIGS. 11 and 12 are calibration curves for used in the determination of gadolinium concentration in human serum samples.

FIG. 13 is a calibration curve created using both MAGNEVIST® and ICP standards.

### DETAILED DESCRIPTION

The invention relates to a method for detecting gadolinium chelates in biological samples. In one aspect of the invention, the chelates can be detected without a chromatographic separation step to separate the chelates from endogenous compounds in biological samples prior to the detection of gadolinium. The detection of gadolinium chelates in biological samples allows for the determination of glomerular filtration rate in animals. Following the administration of a gadolinium chelate to an animal, the level of the chelate in biological samples taken from the animal at various intervals can be correlated to glomerular filtration rate. In various aspects of the invention, the convenience, availability and inexpensiveness of the method is enhanced when the method employs a single stable liquid reagent which can be readily utilized by high-throughput automated analyzers common to most modern clinical laboratories.

As used herein, the singular forms “a,” “an”, and “the” include plural referents unless the context clearly dictates otherwise.

Gadolinium chelates can be detected in various biological samples. A “sample” is an aliquot of any matter containing, or suspected of containing, a gadolinium chelate. Biological samples include all samples from taken from animals (e.g., tissue, hair and body fluids such as serum, plasma, saliva urine, tears and pleural, spinal or synovial fluids). While, in one of aspect the invention, the chelates are detected without separating the chelates from endogenous compounds in biological samples, it may be appropriate to conduct routine clinical preparation of the sample prior to detecting the chelates. For example, whole anticoagulated blood may be centrifuged to provide a plasma sample, or allowed to clot prior to centrifugation to produce serum samples. Various anticoagulants include lithium-heparin, EDTA, oxalate, citrate and fluoride-oxalate. Where the sample is initially complex, solid, or viscous, it can be extracted, dissolved, filtered, centrifuged, stabilized, or diluted in order to obtain a sample having the appropriate characteristics for use with the invention. For the purposes herein, “sample” refers to either the raw sample or a sample that has been prepared or pre-treated. It is not necessary, however, to perform HPLC on a sample prior to detecting gadolinium with the method of the invention.

A number of commercially available gadolinium chelates are available and detectable in biological samples. These chelates include MAGNEVIST® brand (Berlex Laboratories, Montville, N.J.) of gadopentetate dimeglumine injection, which is the N-methylglucamine salt of the gadolinium complex of diethylenetriamine pentaacetic acid (DTPA), and is an injectable contrast medium for magnetic resonance imaging (MRI). Other commercially available gadolinium chelates represent analogues of gadolinium-DTPA and include gadoversetamide (OPTIMARK®; Mallinckrodt Medical), gadoteridol (Prohance; Bracco), and gadodiamide (OMNISCAN™; Amersham Health). In addition, reagent grade gadolinium-DTPA is available from Sigma-Aldrich.

Detection of the gadolinium chelate in a biological sample includes contacting the sample with a dye that is reactive with gadolinium at a pH of about 1.0 to about 4.0. At this pH, the gadolinium binds far more strongly to the dye than to the chelating agent, which produces a color change that can be detected spectrophotometrically.

Arsenazo III is a dye that forms a colored complex with gadolinium in an acidic solution at about pH 2 to about pH 4. The optimum absorbance for analysis of solutions containing this complex occurs at a wavelength in the range of about 600 to 680 nanometers. Chlorophosphonazo can also produce a significant result, generally at a pH of about 1.0 to about 3.0, although the high absorbance of its uncomplexed form limits its range and precision relative to arsenazo III.

In one aspect, the method of the invention includes detecting gadolinium at a pH of about 1.0 to about 4.0. The desired pH range for detecting gadolinium chelates with arsenazo III and chlorophosphonazo has been determined empirically. Accordingly, small variations in outer limits of the range are expected and within the scope of the invention. At this pH the commercially available gadolinium chelates preferentially release the gadolinium cation to the dye. Accordingly, an appropriate buffer should maintain the reaction mixture in that pH range. In other aspects, the pH range for detection of gadolinium with arsenazo is about 2.0 to about 3.0 and more specifically, about 2.2 to about 2.8. A non-exhaustive list of low-pH suitable buffering systems that would not strongly chelate gadolinium are provided in Table 1.

TABLE 1

| Weak Acid | Acid pKa |
|---|---|
| Bisulfate | 1.96 |
| Maleic acid | 2.00 |
| Glycine | 2.35 |
| Diglycolic acid | 2.96 |
| Malonic acid | 2.88 |
| Diglycine | 3.14 |
| 3,3-Dimethylglutaric acid | 3.70 |
| Glycolic acid | 3.83 |
| Barbituric acid | 4.04 |
| Fumaric acid | 3.03, and 4.38 |
| Succinic acid | 4.2, and 5.6 |

In certain embodiments of the invention, the weak acid upon which the buffer is based is glycine, bicine (N,N-bis(2-hydroxyethyl)glycine) or tricine (N-(2-hydroxy-1,1-bis(hydroxymethyl)ethyl)glycine). The effective pKs of the 0.1 M glycine-sulfate, bicine-sulfate and tricine-sulfate buffers were measured to be about 2.57, 2.17 and 2.31 respectively. Tricine is less likely to be degraded by microbes than glycine, and can form buffers with effective pKs near the optimal 2.4 for arsenazo III and within the appropriate range for chlorophosphonazo. In other embodiments of the invention, the buffer is based on 1,2,4-triazole. The triazole-sulfate buffer has an effective pK of about 2.3.

In one embodiment of the invention, the buffer includes sulfate. Buffers having sulfate counterions can provide higher sensitivity and/or lower background signal than those of corresponding buffers having chloride or cyanoacetate counterions.

The use of so-called “self buffering” systems can reduce the temperature dependency of the assays of the present invention. Sulfate has buffering properties of its own, and sulfate buffers have a positive dpK/dT. Accordingly, when sulfuric acid is used in glycine, bicine, tricine or triazole-based buffers, it can help offset their negative dpK/dTs. Tricine-sulfate and triazole-sulfate buffer systems have low enough temperature dependence that there is little difference in assay results over a temperature range of 22-37° C.

Moreover, the tricine-sulfate, bicine-sulfate, triazole-sulfate and glycine-sulfate buffers suitable for use in the present invention are also double buffers. “Double buffers,” as used herein, refers to a buffer system having two distinct buffering components (e.g., tricine and sulfate) having pKa values within 0.5 pH units of one another. When one buffer component is a counterion to another buffer component, as sulfate is to glycine, greater buffering capacity can be achieved at relatively lower buffer concentrations. High ionic strength buffers can have a deleterious effect on the assay methods of the present invention; accordingly, in one aspect of the invention, the use of double buffers can provide reagents with relatively low ionic strengths.

According to one aspect of the invention, a tricine-sulfate buffer comprises an aqueous solution of tricine and sulfate, each in the concentration range of about 0.02 M to about 1 M (summed over all protonation states). The buffer can have, for example, a pH in the range of about 2.0 to about 4.0. In certain embodiments of the invention, the concentrations of tricine and sulfate are in the concentration range of about 0.07 M to about 0.5 M. In further embodiments of the invention, the concentrations of tricine and sulfate are in the concentration range of about 0.1 M to about 0.3 M. The skilled artisan can use standard buffer formulation techniques to prepare the tricine-sulfate buffers of the present invention.

According to another aspect of the invention, a triazole-sulfate buffer comprises an aqueous solution of triazole and sulfate, each in the concentration range of about 0.02 M to about 1 M (summed over all protonation states). The buffer can have, for example, a pH in the range of about 2.0 to about 4.0. In certain embodiments of the invention, the concentrations of triazole and sulfate are in the concentration range of about 0.07 M to about 0.5 M. In further embodiments of the invention, the concentrations of triazole and sulfate are in the concentration range of about 0.1 M to about 0.3 M. The skilled artisan can use standard buffer formulation techniques to prepare the triazole-sulfate buffers of the present invention.

Standard techniques can be used to formulate buffer systems. Other components used in the assays (e.g., chelators and alkylsulfonic acids, described below) may need to be accounted for in determining buffer component concentrations. More information on buffer systems, including self buffering systems, can be found in D. D. Perrin and Boyd Dempsey, “Buffers for pH and Metal Ion Control,” Chapman and Hall Publishers, 1974, which is hereby incorporated by reference.

The optimum pH for detecting gadolinium with arsenazo is about 2.4. Chlorophosphonazo has a more acidic optimum, pH of about 1.0 to about 2.0, rather than 2.4 for arsenazo, and although its sensitivity is comparable to that of arsenazo it produces much higher nonspecific absorbance. In another aspect of the invention, the pH range for detection of gadolinium with chlorophosphonazo is about 1.5 to about 2.5.

The reaction for either dye is not very selective. All elements reacting with the dyes produce nonspecific absorbance and/or act as inhibitors in the presence of gadolinium ion. Although a number of metal ions are known to interfere with traditional methods for detection of gadolinium, few of these are significantly present in biological samples, except iron and calcium. Calcium is well known to bind strongly to arsenazo, and can produce high nonspecific color in samples when measuring gadolinium. In addition, the level of serum calcium is higher than that of gadolinium after administration of the standard dose (0.1 mmol/kg) of gadolinium, which prevents binding of gadolinium to the arsenazo detection reagent. Accordingly, traditional methods for detecting gadolinium have removed these ions from the samples, for example by HPLC, prior to the determination of gadolinium using arsenazo. The use of low pH in the present method of detecting gadolinium in plasma or serum mitigates the interference of ferric and calcium ions, while at the same time producing maximal sensitivity and thereby avoiding the need for HPLC.

Nevertheless even at low pH, calcium and ferric ions produce interference that significantly limits the precision and range of the gadolinium assay response. For instance, while calcium interference decreases exponentially with pH, it is not completely eliminated. In addition, the affinity of gadolinium for arsenazo dye decreases with pH, reducing both linearity and sensitivity of gadolinium response. In one aspect, the method of the invention allows for a pH window where the pH is high enough to allow highly efficient measurement of gadolinium chelate while reducing calcium interference by 99% relative to its maximal binding to Arsenazo at pH 6. Nonetheless, even within this optimally selective pH window, interference from both calcium and ferric ions is substantial. To remedy this persistent residual calcium interference, in another aspect of the invention, the compound HDMP, commonly used as an oral iron chelator for treatment of thalassemia (iron overload), is added to the reagent of the invention to effectively mask interference from both calcium and ferric ions without substantially reducing gadolinium assay response.

In general, chelating agents, including EDTA, EGTA, TTHA, EDTPO, phenanthroline, and 8-hydroxyquinoline have a greater affinity for rare earth metals, such as gadolinium, than for calcium. HDMP, however, is unusual in its ability to bind calcium preferentially over gadolinium. The use of an optimal amount of HDMP can achieve greater than 90% reduction in calcium interference at pH 2.4 with less than 10% reduction in gadolinium signal. This essentially, although not completely, eliminates the interference by calcium with the method of the invention. Other analogs of HDMP, particularly derivatives of hydroxypyridone or hydroxypyrone and possessing an aromatic alpha-hydroxy ketone motif can reasonably be expected to be of similar utility as HDMP for preferential chelation of calcium in the presence of gadolinium. However, maltol (3-hydroxy-2-methyl-4-pyrone), a close analog of HDMP, has no effect on reducing calcium interference.

In another aspect of the invention, the sample is contacted with an alkylsulfonate (either as the free acid or its conjugate base) having a critical micelle concentration (CMC) in the range of 1-100 mM. For example, in one embodiment of the invention, the alkylsulfonate is a linear C₄-C₈ alkylsulfonate. A linear C₄-C₈ alkylsulfonate is an alkylsulfonate in which the alkyl group is linear and 4-9 carbons in length. In other embodiments of the invention, the alkylsulfonate is a branched alkylsulfonate or a cycloalkylsulfonate having a CMC in the range of 1-100 mM. In certain embodiments of the invention, the alkylsulfonate is mixed with the sample at the same time as the dye. Alternatively the alkylsulfonate can be mixed with the sample at a different time than the dye, but before the measurement step. Alkylsulfonates are commonly used as mobile phase additives in reverse phase HPLC, in which they bind electrostatically to polar analytes and enhance the interaction with reverse phase adsorbents, thereby improving separation. The use of alkylsulfonates (e.g., linear C₄-C₈ alkylsulfonates) in the present invention can reduce nonspecific color response, which can be a complication for certain samples (especially feline samples). Without being bound by this theory, the inventor believes that this nonspecific color response is due to interaction of the dye with certain high molecular weight components, such as albumin. C₄-C₈ alkylsulfonates reduce calorimetric interference with roughly the same chain length dependence as for their critical micelle concentrations. Longer linear alkylsulfonates tended to increase turbidity due to micelle formation and precipitation. While the entire range of linear C₄-C₈ alkylsulfonates is suitable for use in the present invention, linear C₆-C₈ alkylsulfonates do not suffer substantially from wetting issues, and relatively low concentrations are necessary to achieve reduction of calorimetric interference. In one embodiment of the invention, the alkylsulfonate is a linear C₇ alkylsulfonate. Alkylsulfonates can be provided, for example, using alkylsulfonic acids or their corresponding salts. Examples of suitable salts include alkali metal salts (e.g., sodium and potassium) and ammonium. In certain embodiments of the invention, the alkylsulfonate is present during the contacting at a concentration below its CMC (e.g., by at least 1 mM, at least 2 mM, or at least 5 mM).

Reagents suitable for use in performing the methods of the present invention are described in more detail below.

In one aspect of the invention, the various chelated forms of the gadolinium, including metabolized (e.g., hydrolyzed, conjugated) or other bound forms (e.g., complexes of gadolinium with transferrin, citrate, or albumin) are not separated prior to measurement of total gadolinium. Instead of measuring only one form or another, total gadolinium is measured. For measurement of GFR this is an advantage relative to other more specific methods, such as HPLC or immunoassay which could produce variable results as the form of the chelated gadolinium changes depending on the age, stability and other variable characteristics of the sample.

The method of the invention includes contacting a biological sample with a dye at a low pH. In the most basic aspect of the invention, the dye is buffered in solution at the appropriate pH and the sample is contacted with the dye by forming a mixture of the sample and the dye solution. The solution is maintained within the appropriate pH with a suitable buffer. The absorbance of the solution is measured and the color or the change in color of the solution can be detected and compared to known standards. A suitable calibration curve based upon various concentrations of chelated gadolinium can be prepared.

In general, glomerular filtration rate (GFR) can be measured by dosing an animal with a GFR marker and measuring its blood clearance. Animal volume distribution kinetics are three times faster in cats and dogs than humans. For example, distribution half-life is about 5 minutes in cats and dogs versus 15 minutes for humans. Thus, animals are typically sampled at 30, 60, and 90 minutes after infusion of the GFR marker, whereas human subjects are usually sampled at 120, 180, and 240 minutes.

When compared to ICP-MS, which is known as the “gold standard” method of detecting gadolinium, the present method showed as little as a 2% difference between clearance rates obtained by each method, which is within the margin of assay imprecision (each method has a precision of about 2% CV). While neither feline serum nor plasma samples produce any significant turbidity, canine plasma samples obtained using fluoride-oxalate, lithium-heparin or potassium EDTA anticoagulants produce significant turbidity. Human plasma is reported to produce turbidity with reagents of similar pH due to acid precipitation of fibrinogen or fibrin. Serum is thus preferred for measurement of GFR.

The precision of the method for GFR is particularly important since changes are progressive over many years and intervention is most effective when applied before irreversible damage occurs, resulting in renal failure requiring treatment by dialysis or transplant. For example in one prospective study of about 50 diabetic patients monitored yearly by GFR, many exhibited a steady progressive decrease in GFR of 5-10% per year, marked by an occasional renal crisis often followed by a return to steady decline. The high precision and reproducibility of the method (1-2% CV) of the invention, typical for other automated clinical chemistries such as glucose, protein and calcium, can reasonably be expected to discern yearly changes of this magnitude (5-10%), allowing timely therapeutic intervention in patients with chronic progressive nephropathy. It has been established that glycemic control and antihypertensive therapy can halt or reverse the progression of nephropathy.

In one aspect, the invention is directed to a reagent for detecting a gadolinium chelate in a biological sample. As used herein, “reagent” refers to a substance that participates in a chemical reaction or physical interaction. A reagent can comprise an active component, that is, a component that directly participates in a chemical reaction and other materials or compounds directly or indirectly involved in the chemical reaction or physical interaction. It can include a component inert to the chemical reaction or physical interaction, such as catalysts, stabilizers, buffers, and the like.

The reagent of the invention includes a dye selected from the group consisting of arsenazo III and chlorophosphonazo, and a buffer for maintaining the reagent at a pH from about 2.0 to about 4.0 when the dye is arsenazo III, or at a pH from about 1.0 to about 3.0 when the dye is chlorophosphonazo. Suitable buffers are discussed above and should be used in amounts effective to maintain the buffer capacity of the reagent in light of the amount of sample. Either Arsenazo III or chlorophosphonazo is generally used in an amount from about 100 μM to about 1.0 mM, or in particular, from about 200 μM to about 500 μM.

In one aspect of the invention, the reagent includes a glycine-sulfate, tricine-sulfate, bicine-sulfate or triazole-sulfate buffer as described above. In one embodiment of the invention, the buffer is a tricine-sulfate buffer. In another embodiment of the invention, the buffer is a triazole-sulfate buffer.

In one aspect of the invention, the reagent includes HDMP. For example, a reagent of the invention can contain about 10 to about 2000 mM HDMP. In various aspects, the reagent contains about 10 to about 800 mM HDMP, and more particularly about 70 mM HDMP. In one aspect, 70 mM HDMP masks 87% of the calcium and greater than 95% of ferric ion without significant effect on gadolinium response. Higher levels of HDMP may be selected to further remove calcium and iron interference based upon analytical sensitivity, matrix effects (e.g., diet, drugs, toxicants, lipemia and icterus), solubility, sample quality (e.g., hemolysis) and storage stability.

In one embodiment of the invention, the reagent includes an alkylsulfonate having a CMC in the range of 1-100 mM. As described above, the alkylsulfonate can be provided as the acid or in a salt form. In certain embodiments of the invention, the alkylsulfonate is a linear C₄-C₉ alkylsulfonate. For example, the linear C₄-C₉ alkylsulfonate can be a linear C₆-C₈ alkylsulfonate. In other embodiments of the invention, the alkylsulfonate is a branched alkylsulfonate or a cycloalkylsulfonate having a CMC in the range of 1-100 mM. The concentration of alkylsulfonate can be, for example, in the range of about 1 to about 100 mM. In certain embodiments of the invention, the concentration of alkylsulfonate is below its CMC (e.g., by at least 1 mM, at least 2 mM, or at least 5 mM). For example, when the alkylsulfonate is sodium hexanesulfonate, concentrations can be, for example, in the range of 35-55 mM (e.g., about 45 mM). When the alkylsulfonate is sodium heptanesulfonate, concentrations can be, for example, in the range of 5-25 mM (e.g., about 16 mM). When the alkylsulfonate is sodium octanesulfonate, concentrations can be, for example, in the range of 2-10 mM (e.g., about 5 mM).

The reagents of the present invention may also include other additives, such as a nonionic surfactant. In one embodiment of the invention, the reagent includes the nonionic surfactant TRITON® X-100 (e.g., (CH₃)₃C—CH₂—(CH₃)₂C-Ph-O—(CH₂CH₂O)ₓH, in which x9.5). When a nonionic surfactant such as TRITON® X-100 is present in the reagent, it can have a concentration of 0.01% to about 1%. For example, the concentration of TRITON® X-100 can be about 0.2%.

Another aspect of the invention is a kit for use in a spectrophotometric method for determining gadolinium concentration in serum-free samples or in stock solutions containing free or chelated gadolinium. The kit comprises a reagent as described above, as well as at least one standard solution of a known gadolinium concentration. Each standard solution can include chelated gadolinium (e.g., Gd-DTPA) or free (i.e., not chelated) gadolinium. Free gadolinium solutions having very well-defined concentrations are available commercially, for example, from Sigma-Aldrich, Milwaukee, Wis. as catalog no. 356220, “Gadolinium ICP/DCP standard solution.” Standard solutions having free gadolinium are typically supplied as strong acid solutions (e.g., 0.1-10% nitric acid), while standard solutions having chelated gadolinium are supplied as weakly acidic, neutral, or weakly basic solutions (e.g., pH 4-10). The standard solutions can vary in gadolinium concentration, for example, in the range of 0 to about 2 M. In certain embodiments of the invention, the standard solutions vary in concentration, in the range of 0 mM to 1 M. In certain embodiments of the invention, the calibration samples vary in concentration from 0 M to about 600 μM. In one embodiment of the invention, the gadolinium species in the one or more standard solutions is a chelated gadolinium species, such as a gadopentetate species, a gadodiamide species, a gadoversetamide species or a gadoteridol species. Standard solutions can be used in the calibration as supplied, or can be diluted to yield a calibration sample having an appropriate gadolinium concentration.

In certain embodiments of the invention, especially for calibration samples having concentrations in the range of about 1-1000 μM, the gadolinium species is a free gadolinium ion. At concentrations of about 1-1000 M, free gadolinium ions and chelated gadolinium species provide substantially the same results in the assays described herein. For example, calibration samples can be similar to those used in calibration of ICP-based methods (e.g., free gadolinium ions in 0.025% nitric acid).

In general, the amount of sample should be optimized to avoid interference from compounds in the sample and interferences associated with turbidity; for example the plasma precipitation which becomes a problem at acid pH. In addition, sample preparation methods will affect the amount of sample that can or should be used in method of the invention. The amount of the sample must be enough to provide an accurate determination of an amount of gadolinium in the sample. For serum samples, the amount of the sample should reflect from about 1% to about 50% of the total reaction volume. Sample concentrations as low as 7% have been shown to provide optimal performance in the determination of gadolinium chelates using the method of the invention. In the method of the invention for determination of GFR, an additional constraint is that the range of concentrations must fall within the dynamic range of the assay, e.g., a 7% serum assay volume will allow accurate measurement of approximately 50-1000 μM gadolinium chelate. This range of gadolinium concentrations encloses the range of calculated GFR values exhibited for normal and pathological samples in humans, dogs and cats using a 0.1 mmol/kg dose of MAGNEVIST®. To optimize the dose for GFR measurement in normal and pathological samples, the dose of gadolinium chelate can be adjusted to produce proportional amounts of serum gadolinium.

Turbidity (e.g., from fibrinogen interference) is one common interferent in the gadolinium assays described herein. This type of reagent-induced turbidity is distinct from sample turbidity such as due to lipemia, the more common cause of turbidity in serum chemistry tests. Even very high levels of lipemia are not a significant interference in the methods of the present invention. One method for mitigating the effects of turbidity (and, to a lesser extent, hemolysis), is to use 700 nm as a secondary wavelength (i.e., a wavelength for which the absorbance is subtracted from the signal absorbance to provide a net absorbance value which is used in subsequent calculations). Turbidity can also be corrected for by using a calibration curve. Accordingly, in one embodiment of the invention, the assay method is performed and absorbance measurements are taken at both 654 nm and 750 nm for a variety of serum samples, both unspiked and spiked with fibrinogen (e.g., 0.5-4.5 mg/mL). For each sample, the difference between its net absorbance (A₆₅₄-A₇₅₀) and the net absorbance for the unspiked sample (the excess net absorbance) is plotted vs. its A₇₅₀, and a calibration equation is generated (e.g., by fitting the data to a third order polynomial). For the samples of interest, both A₆₅₄ and A₇₅₀ are measured and a net absorbance calculated. The sample A₇₅₀ value is converted by the calibration equation to an excess net absorbance for the sample, which is subtracted from the net absorbance of the sample to yield an adjusted net absorbance, which is then used with the gadolinium calibration curve to calculate gadolinium concentration.

Hemoglobin (e.g., due to hemolysis) is another common interference in the gadolinium assays described herein. The additive interference caused by hemoglobin is independent of gadolinium concentration, and can be corrected for using a hemoglobin calibration curve. Accordingly, in one embodiment of the invention, serum samples are spiked with hemoglobin at varying concentrations (e.g., 0-3 mM), and are assayed as described above. The increase in measured gadolinium concentration (in μM) is plotted vs. hemoglobin concentration (in mg/mL) to create a hemoglobin calibration curve. The hemoglobin concentration of the sample of interest can be measured at 414 nm or by using bichromatic measurement of the sample at 410 nm (primary wavelength) and 480 nm (secondary wavelength), and the measured gadolinium concentration of the sample can be corrected by subtracting from it the product of the measured hemoglobin concentration (in mg/mL) and the slope of the plot (in μM-mL/mg).

The following are provided for exemplification purposes only and are not intended to limit the scope of the invention described in broad terms above. All references cited in this disclosure are incorporated herein by reference.

### EXAMPLES

### Example 1

### Gadolinium-DTPA Assay in Water

The release of gadolinium from the gadolinium-DTPA complex was measured using the gadolinium-arsenazo III system. The gadolinium-DTPA, 54.8 mg (0.1 mmol) (Sigma-Aldrich, St. Louis, Mo.) was solubilized in 10 mL of water containing 17 mg of NaHCO₃ to produce a 10 mM stock Gd-DTPA. One mL of a reagent solution containing the 100 μM arsenazo III in 20 mM phthalate buffer (Sigma-Aldrich, St. Louis, Mo.), pH 3.0, was mixed with 0.5-12 μL of 10 mM Gd-DTPA stock, producing assay concentrations of Gd ranging from 5-120 μM. FIG. 1 shows a plot of the absorbance of the solution at 656 nm as a function of the gadolinium-DTPA concentration.

### Example 2

### Gadolinium-DTPA Assay in Cat Serum

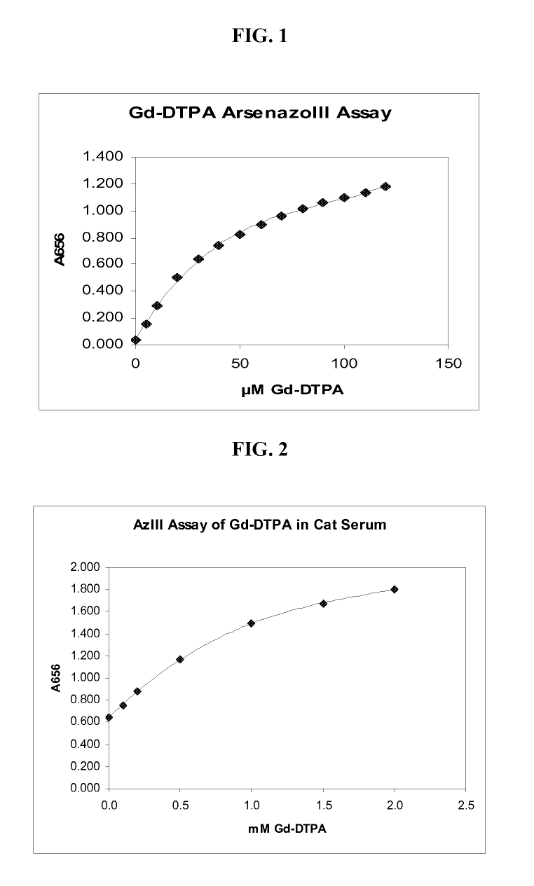

The same experiment was performed as described in Example 1 except that the assay was performed using reconstituted lyophilized cat serum (Sigma). 50 μL of cat serum containing 0.1-2 mM Gd-DTPA was added to 1 mL of reagent containing 0.2 mM arsenazo III in 20 mM phthalate buffer, pH 3.0. FIG. 2 shows the absorbance of the solution at 656 nm at various concentrations of gadolinium-DTPA.

### Example 3

### Gadolinium-DTPA Assay in Canine Serum with Removal of Interfering Cations

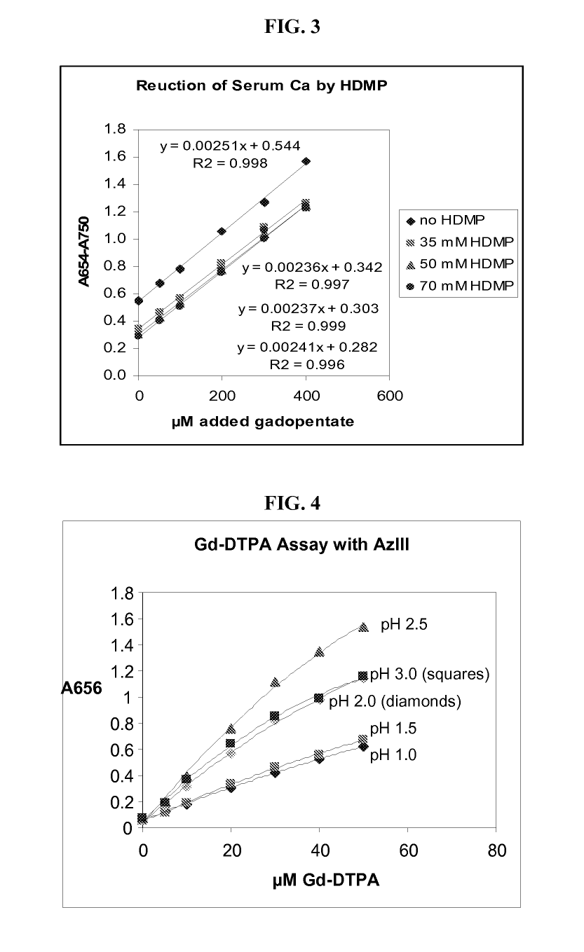

An experiment similar to that of Example 2 was performed except that a canine serum sample assay was spiked with 35, 50 and 70 mM HDMP. 0.93 mL of 350 μM arsenazo III in 0.2 M glycine-sulfate buffer, pH 2.35, was prepared containing the various amounts of HDMP. Commercial Gd-DTPA (MAGNEVIST®, Berlex Laboratories, Wayne N.J.) ranging from 50-400 μM was added to 0.07 mL of canine serum. The canine serum was added to the arsenazo III reagent in varying amounts. FIG. 3 shows the net bichromatic absorbance of each solution. This is obtained by subtracting the absorbance of each solution at 750 nm from its absorbance at 654 nm, reducing interference from wavelength-independent absorbance due to sample turbidity.

To determine the effect of HDMP on removal of ferric ion interference, ferric sulfate in a final concentration of 20 μM was added to 1 mL of reagent containing 250 μM arsenazo III in 0.2 M glycine-sulfate buffer, pH 2.35. The ferric sulfate increased the net absorbance of the solution (A654 nm minus A750 nm). This amount of ferric ion is approximately 10 times the amount that would be contributed by 0.07 mL of a normal canine serum sample. HDMP, even at concentrations much lower than used in the reagent was effective at eliminating almost all of the interference of the ferric ion (data not shown).

### Example 4

### pH Optimization for Gadolinium-DTPA System

The pH was optimized for the gadolinium-DTPA assay. Stock gadolinium-DTPA solution was prepared as described in Example 1. A 150 μM arsenazo III dye solution in a sulfate buffer system was prepared for solutions whose pH values ranged from 1.0 to 2.5, and a phthalate buffer system was prepared for the solution at pH of 3.0. FIG. 4 shows the absorbance at 656 nm for various gadolinium-DTPA concentrations.

### Example 5

### Further Optimization of pH for Gadolinium-DTPA System

Using glycine-sulfate as the buffer, a similar experiment to that of Example 4 was carried over pH ranges of 2.2-2.8. The arsenazo III concentration was 100 μM. Table 2 shows the absorbance at 656 nm for various μL amounts of added 2 mM gadolinium-DTPA at varying pH.

TABLE 2

| μL Gd | μM Gd | pH 2.2 | pH 2.3 | pH 2.4 | pH 2.5 | pH 2.6 | pH. 2.7 | pH. 2.8 |
|---|---|---|---|---|---|---|---|---|
| 0 | 0 | .0396 | .0404 | .0408 | .0426 | .0442 | .0425 | .0442 |
| 1 | 2 | .0803 | .0833 | .0806 | .0829 | .0863 | .0844 | .0845 |
| 2.5 | 5 | .1722 | .1876 | .1938 | .2012 | .1966 | .1934 | .1852 |
| 5 | 10 | .3382 | .3626 | .3792 | .3829 | .3740 | .3602 | .3440 |
| 10 | 20 | .6022 | .6591 | .6845 | .6829 | .6499 | .6087 | .5718 |
| 15 | 30 | .8195 | .8993 | .9061 | .8948 | .8457 | .7949 | .7353 |

The data in Table 2 indicate maximal response of the reagent at a pH between 2.3 and 2.6 (mean of 2.45). However, since serum has significant alkaline buffering capacity, a somewhat lower pH of 2.35 may be used to ensure that sample buffer capacity does not produce an assay pH in excess of 2.45. In addition, pH 2.35 is coincident with the pKa of glycine, producing maximal buffer capacity.

### Example 6

### Tricine and Triazole Sulfate Buffers

The use of tricine-sulfate buffers and triazole-sulfate buffers was tested in the gadolinium-arsenazo III system by creating calibration curves. Reagents used in these experiments had arsenazo III at a concentration of 330 μM, and the indicated buffer at a concentration of 0.2 M a pH of 2.35 as well as any other noted components. Both canine and feline serum samples were spiked with gadolinium-DTPA at a variety of concentrations in the 30-300 μM range, then assayed by mixing 70 μL of the sample with 930 μL of reagent, measuring the absorbance at 654 nm and 750 nm. The slopes reported in Table 3 are determined from a plot of Gd concentration vs. the difference between the two measured absorbances, and have units of μM/(Absorbance units×10⁴). The intercepts reported in Table 3 are determined from the same plot as are the slopes, and have units of (Absorbance units×10⁴).

TABLE 3

| | | canine | canine | feline | feline |
|---|---|---|---|---|---|
| Buffer | Additive | slope | intercept | slope | intercept |
| tricine-sulfate | C₇, 16 mM | 0.0480 | 2179 | 0.0469 | 2241 |
| tricine-sulfate | C₈, 6 mM | 0.0486 | 2144 | 0.0473 | 2205 |
| tricine-chloride | C₇, 16 mM | 0.0434 | 2732 | 0.0427 | 2825 |
| triazole- | none | 0.0421 | 3107 | 0.0396 | 3409 |
| cyanoacetate | | | | | |
| triazole- | none | 0.0442 | 2194 | 0.0450 | 2591 |
| chloride | | | | | |
| triazole-sulfate | none | 0.0421 | 2772 | 0.0390 | 3104 |
| triazole-sulfate | C₇, 6 mM | 0.0465 | 2053 | 0.0455 | 2096 |

C₇ = sodium heptanesulfonate;
C₈ = sodium octanesulfonate

### Example 7

### Alkylsulfonate Additives

The effect of alkylsulfonates in reducing nonspecific color when applied to feline samples was tested in the gadolinium-arsenazo III system. Calibration curves were prepared as described above but over a wider range (20-500 μM) for canine and feline serum samples using 0.2 M tricine-sulfate buffer (pH 2.35) having the noted alkylsulfonate additive. Additionally, data were acquired for blank samples (i.e., [Gd]=0). The slopes reported in Table 4 are determined from a plot of Gd concentration vs. the difference between the two measured absorbances, and have units of μM/(Absorbance units×10⁴). The intercepts reported in Table 4 are determined from the same plot as are the slopes, and have units of (Absorbance units×10⁴). Table 4. The blank values reported in Table 4 are the differences in the two measured absorbances, and have units of (Absorbance units×10⁴).

TABLE 4

| | canine | canine | canine | feline | feline | feline |
|---|---|---|---|---|---|---|
| additive | slope | intercept | blank | slope | intercept | blank |
| C₇, 16 mM | 0.0400 | 1817 | 1970 | 0.0392 | 1915 | 2062 |
| C₈, 6 mM | 0.0401 | 2313 | 2465 | 0.0408 | 2430 | 2552 |
| none | 0.0402 | 2460 | 2525 | 0.0414 | 2884 | 2967 |

C₇ = sodium heptanesulfonate;
C₈ = sodium octanesulfonate

### Example 8

### Variation of Analyte Solution Concentration

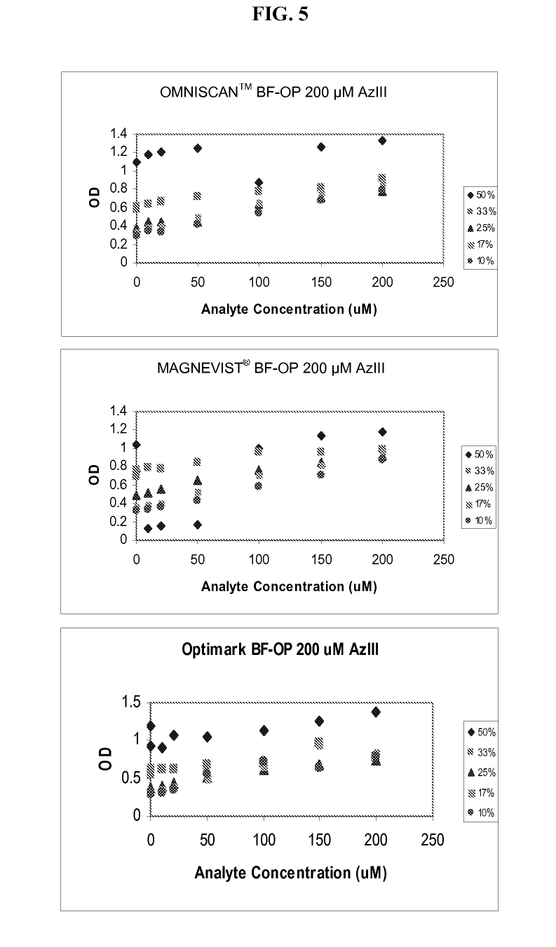

The release of gadolinium from the gadolinium-DTPA complex was tested in the gadolinium-arsenazo III system. Measurements were taken in bovine oxalate plasma (BOP), bovine fluoride-oxalate plasma (BF-OP), bovine serum (FBS), and a buffered reagent. These bovine plasma and serum materials were provided by Rockland Immunochemicals, Inc., Gilbertsville, Pa. Amounts of arsenazo were added to the reagents to achieve final assay concentrations of 50, 100, and 200 μM arsenazo III, buffered at pH 2.45 using a glycine-sulfate buffer. Three different commercial gadolinium chelate agents were tested: MAGNEVIST® (gadopentate), OMNISCAN™ (gadodiamide), and OPTIMARK® (gadoverstetamide). The percentage of the sample in the assay was varied from 50% down to 10%. FIG. 5 shows the data for the three types of DTPA and BF-OP. Best results are produced at lower sample concentrations, e.g. 10%. Infusion of gadolinium chelates for measurement of GFR, which can produce gadolinium concentrations in plasma ranging from about 20-1000 μM, produced optimal sensitivity over a wide range using a sample concentration of 7% (data not shown).

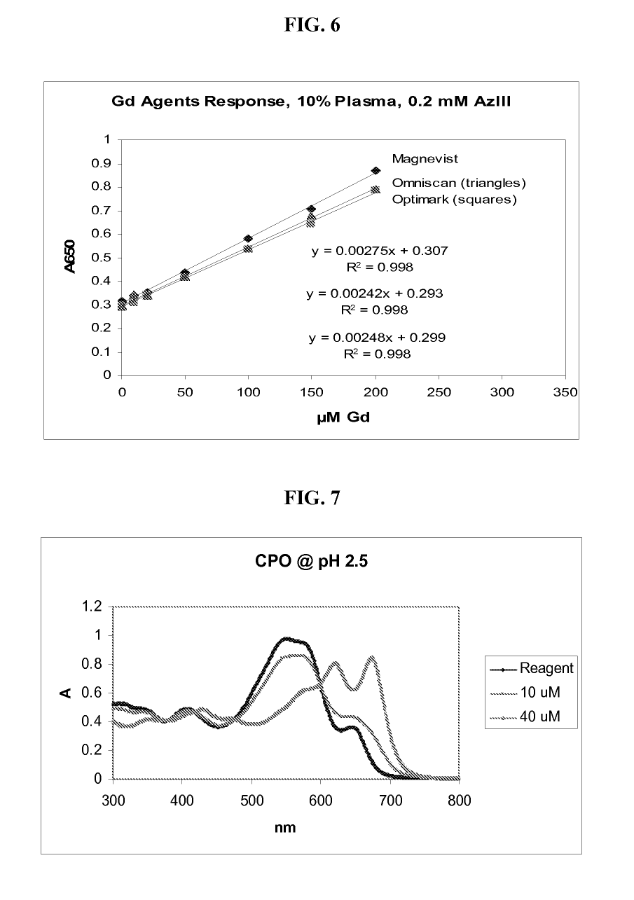

FIG. 6 shows that the BF-OP sample produces strong correlation and sensitivity for all three commercially available DTPA chelates. In this experiment, the arsenazo III concentration is 250 μM in glycine-sulfate buffer at a pH of 2.45. Similar results were achieved with samples of BOP and FBS.

### Example 9

### Comparison to Alternative Dye Systems

Chlorophosphonazo was used as an alternative to arsenazo III as the dye for detecting gadolinium. FIG. 7 shows the absorption spectra of chlorophosphonazo and chlorophosphonazo in a solution of chlorophosphonazo with 10 and 40 μM gadolinium-DTPA at pH 2.5.

### Example 10

### Calibration Linearity with Varying Arsenazo III

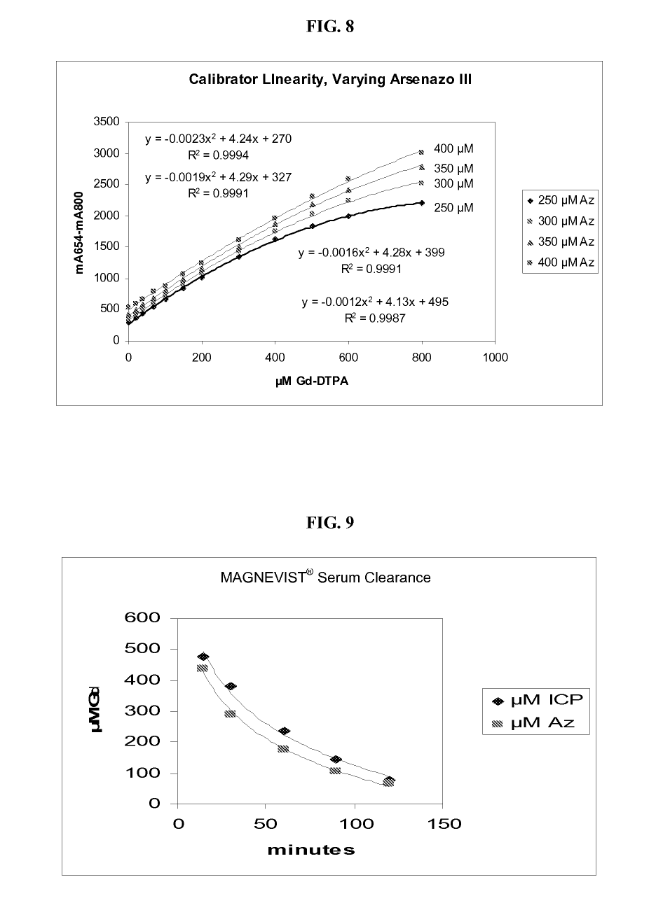

The gadolinium calibrator linearity was tested with bovine plasma using reagents prepared with various levels of arsenazo III. FIG. 8 shows the results using a series of concentrations of gadolinium-DTPA (MAGNEVIST®) at arsenazo III levels of 250 to 400 μM with the system buffered at pH 2.45 using a glycine-sulfate buffer. Although linearity increases with arsenazo concentration, the reagent absorbance also increases. For samples containing amounts of gadolinium ranging from 50-500 μM, sufficient linearity is achieved with minimal background by using an arsenazo III concentration of approximately 350 μM.

### Example 11

### Determination of Glomerular Filtration Rate (GFR) in Dogs

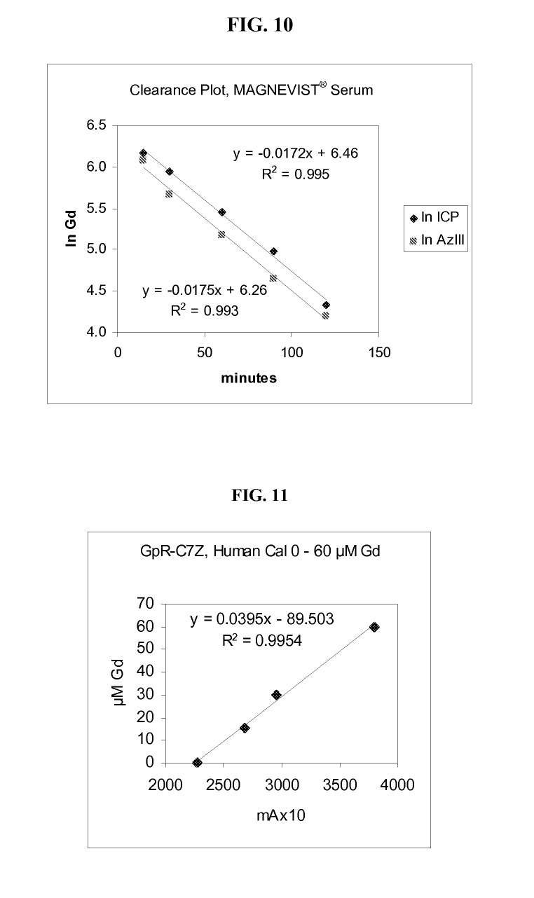

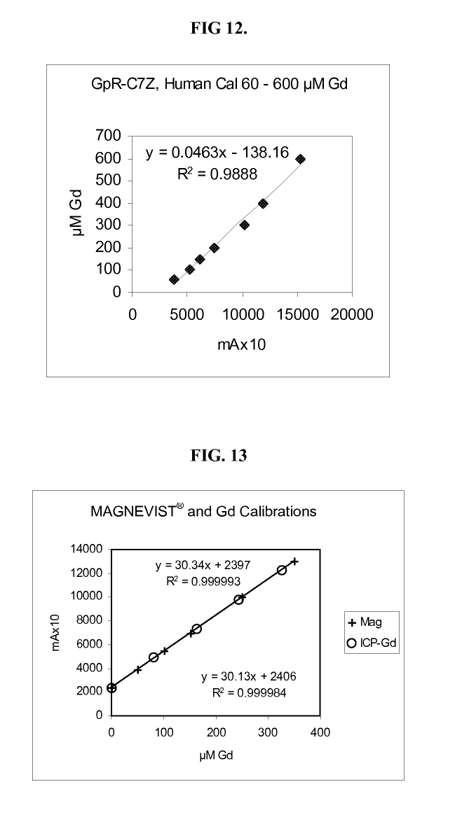

Using an indwelling catheter, a dog was injected intravenously with 0.1 mmol/kg of gadolinium-DTPA (MAGNEVIST®) and samples were taken of dog serum collected at 15, 30, 60, 90 and 120 minutes post injection. As a standard for comparison, the gadolinium concentration of the plasma was measured by ICP-MS (University of Idaho Analytical Services, Moscow, Id.). The gadolinium concentration in the serum was determined according to a method of this invention: to 930 μL of 270 μM arsenazo III in 0.2 M glycine-sulfate, pH 2.40, was added 70 μL of serum or fluoride-oxalate plasma collected from dogs at various times after infusion of 0.1 mmol/kg of gadolinium chelates. Absorbance was determined bichromatically at 654 and 800 nm, and gadolinium concentration of the canine sera and plasma was calculated from the regression line of calibration plots using pooled canine serum or fluoride-oxalate plasma spiked with 20-500 μM of one of the 3 gadolinium chelates. FIG. 9 shows the comparison of the gadolinium concentrations measured in the serum by this method and the gadolinium concentrations measured in the plasma by ICP-MS. FIG. 10 shows the logarithmic plot of gadolinium concentration against time for ICP and AzII-based methods of detecting gadolinium in serum. The slope of this plot yields the clearance rate. GFR can be calculated from the clearance rate by applying the volume distribution obtained from the intercept of the clearance plot and the applied dose of gadolinium chelate. Since the volume distribution is usually constant it has been shown that in most cases use of simple clearance rates or clearance half-life is clinically equivalent and of perhaps superior accuracy and precision to GFR for monitoring progression of disease.

Table 5 shows a comparison of the AzII-based method to the ICP method for dog serum and plasma, and three brands of gadolinium-DTPA chelates. The bias is the absolute difference between the method of the invention and the reference method (ICP-MS); this is also expressed as % of the mean of the two methods (right column).

TABLE 5

| | | Clearance | | | |
|---|---|---|---|---|---|
| | | Slope | | | |
| Sample Type | Sample | ICP | AzIII | Bias | % Difference |
| MAGNEVIST ® | Dog 1 | −0.0152 | −0.0163 | 0.0011 | 7.0 |
| Plasma | | | | | |
| MAGNEVIST ® | Dog 2 | −0.0172 | −0.0176 | 0.0004 | 2.3 |
| Serum | | | | | |
| OMNISCAN ™ | Dog 3 | −0.0140 | −0.0293 | 0.0153 | 70.7 |
| Plasma | | | | | |
| OMNISCAN ™ | Dog 4 | −0.0170 | −0.0173 | 0.0003 | 1.7 |
| Serum | | | | | |
| OPTIMARK ® | Dog 5 | −0.0167 | −0.0223 | 0.0056 | 28.7 |
| Plasma | | | | | |
| OPTIMARK ® | Dog 6 | −0.0152 | −0.0175 | 0.0023 | 14.1 |
| Serum | | | | | |
| OPTIMARK ® | Dog 6* | −0.0174 | −0.0183 | 0.0009 | 5.0 |
| Serum | | | | | |

*120 min time point removed

The results indicate that in canine serum, MAGNEVIST® and OMNISCAN™ more closely approximate the ICP-MS method than the other two chelates as a GFR marker using the method of the invention. The decreased yield for all chelates in fluoride-oxalate plasma is probably due to a specific effect of the anticoagulant and does not rule out plasma sampling using other anticoagulants such as EDTA or heparin. The results using gadoversetamide probably reflect the much slower release of gadolinium from this agent under the assay conditions of the invention. This effect can be mitigated using alternate calibration and longer assay incubation times (5-10 minutes instead of 3-30 seconds), enabling more efficient assay of gadoversetamide by the method of the invention.

### Example 12

### Determination of Gadolinium Concentration in Human Samples

Calibration curves over 0-60 μM and 60-600 μM gadolinium concentration ranges were created by spiking a pooled (n=10) sample of human serum with various concentrations of gadolinium-DPTA and running assays using a reagent having 270 μM arsenazo III and 16 mM sodium heptanesulfonate in 0.2 M triazole-sulfate, pH 2.40, was added 70 μL of serum. 70 μL of serum was added to 930 μL of reagent, and absorbances measured at 654 nm and 750 nm. Calibration curves are shown in FIGS. 11 (0-60 μM) and 12 (60-600 μM). For 0-60 μM, the slope was 28.9 (Absorbance units×10⁴)/μM and the intercept was 2197 (Absorbance units×10⁴); for 60-600 μM, the slope was 23.1 (Absorbance units×10⁴)/μM and the intercept was 2853 (Absorbance units×10⁴).

Twenty random human serum samples from Interstate Blood Bank were spiked with gadolinium-DPTA at 50, 200 and 600 μM. The gadolinium concentrations in the sera were determined according to the same assay method used to determine the calibration curves. Table 6 shows the characteristics of the serum samples, including race (White, Black or Latino), gender (Male or Female) and age of the patient, relative turbidity (on a scale of 1-4 pluses), and absorbance measurements of the unadulterated samples at 654, 700 and 750 nm. Table 7 shows measured data for the spiked samples.

TABLE 6
Interstate Blood Bank human serum samples M40 65296

| | | | Age | Visual | | | |
|---|---|---|---|---|---|---|---|
| Patient | Race | Gender | (y) | Turbidity | A654 | A700 | A750 |
| 1 | W | F | 21 | + | −0.0010 | −0.0015 | −0.0018 |
| 2 | W | M | 36 | ++ | 0.0241 | 0.0186 | 0.0140 |
| 3 | B | M | 19 | +++ | 0.2127 | 0.1821 | 0.1552 |
| 4 | B | M | 37 | + | 0.0064 | 0.0080 | 0.0075 |
| 5 | B | M | 19 | + | 0.0064 | 0.0049 | 0.0034 |
| 6 | B | M | 35 | ++++ | 0.0448 | 0.0399 | 0.0357 |
| 7 | B | M | 36 | + | 0.0077 | 0.0071 | 0.0067 |
| 8 | B | M | 52 | +++ | 0.0794 | 0.0659 | 0.0561 |
| 9 | B | M | 41 | ++++ | 0.0576 | 0.0481 | 0.0400 |
| 10 | B | M | 53 | + | −0.0016 | −0.0034 | −0.0031 |
| 11 | B | M | 37 | ++ | 0.0195 | 0.0170 | 0.0152 |
| 12 | B | M | 21 | +++ | 0.1362 | 0.1181 | 0.1022 |
| 13 | B | M | 41 | + | 0.0301 | 0.0250 | 0.0210 |
| 14 | L | M | 30 | ++ | 0.0203 | 0.0179 | 0.0160 |
| 15 | B | M | 54 | + | 0.0044 | 0.0036 | 0.0019 |
| 16 | B | M | 46 | ++++ | 0.0273 | 0.0222 | 0.0179 |
| 17 | B | M | 28 | ++++ | 0.0551 | 0.0455 | 0.0368 |
| 18 | B | M | 53 | + | −0.0034 | −0.0040 | −0.0043 |
| 19 | B | M | 40 | + | 0.0085 | 0.0060 | 0.0044 |
| 20 | L | M | 42 | + | −0.0020 | −0.0026 | −0.0029 |

TABLE 7

| Patient | 0 μM Gd | 50 μM Gd | 200 μM Gd | 600 μM Gd |
|---|---|---|---|---|
| 1 | −3.0 | 44.7 | 198.7 | 599.5 |
| 2 | −2.4 | 48.0 | 213.4 | 570.8 |
| 3 | −2.6 | 42.0 | 204.8 | 573.1 |
| 4 | −2.8 | 43.4 | 200.5 | 589.1 |
| 5 | −3.0 | 45.6 | 192.2 | 598.1 |
| 6 | −3.3 | 46.3 | 206.0 | 591.5 |
| 7 | −2.6 | 42.7 | 195.1 | 561.4 |
| 8 | −2.3 | 44.8 | 200.2 | 611.4 |
| 9 | −1.2 | 50.6 | 213.5 | 608.2 |
| 10 | −4.4 | 43.6 | 203.6 | 581.5 |
| 11 | −1.6 | 47.0 | 210.5 | 582.2 |
| 12 | −1.8 | 46.5 | 210.1 | 599.1 |
| 13 | −2.7 | 46.2 | 187.0 | 593.7 |
| 14 | −3.2 | 47.4 | 199.5 | 487.1 |
| 15 | −2.6 | 46.3 | 209.0 | 594.5 |
| 16 | −2.6 | 45.4 | 212.4 | 594.2 |
| 17 | −0.9 | 46.8 | 214.3 | 600.0 |
| 18 | −2.4 | 46.9 | 209.0 | 594.9 |
| 19 | −1.7 | 46.3 | 205.2 | 593.7 |
| 20 | −1.6 | 46.3 | 209.6 | 588.5 |
| AVG | −2.4 | 45.8 | 204.7 | 585.6 |
| SD | 0.8 | 2.0 | 7.6 | 26.2 |
| % CV | −33.1 | 4.3 | 3.7 | 4.5 |

The experiment was repeated using a reagent similar to that described above, but also including 0.2 wt % TRITON X-100. When the reagent included the TRITON X-100, variability at the 600 μM measurement was greatly reduced (standard deviation=7.6 μM).

### Example 13

### Reagent Formulation

An example of a buffered arsenazo reagent suitable for use in the present invention is an aqueous solution of 0.2% TRITON X-100, 0.3 mM arsenazo III and 16 mM sodium heptanesulfonate in 0.2 M tricine-sulfate buffer adjusted to pH 2.4.

An example of a calibration sample can be prepared as follows: To 150 mL bovine serum is added 66 μL of 2.5 mg/mL aqueous PPACK (i.e., D-Phe-Pro-Arg-chloromethylketone). After 24-72 hours incubation at 4 C, 150 μL PROCLIN 950 (e.g., 9.5% total 5-Chloro-2-methyl-4-isothiazolin-3-one and 2-methyl-4-isothiazolin-3-one in dipropylene glycol) is added. The mixture is filtered through a 0.2μ filter, and MAGNECAL is added to provide a desired concentration of gadolinium.

### Example 14

### Correction for Interference

All absorbances in this example are reported in units of (Absorbance units×10⁴).

To create a calibration curve for correction of hemoglobin, assays as described above were performed on serum samples having various gadolinium concentrations (0-150 μM) and various hemoglobin concentrations (0-2 mM). The mean increase in measured gadolinium concentration was plotted vs. hemoglobin concentration. The resulting line had the following equation: [Gd]=(4.969 μM·mL/mg)[Hgb]−0.13 μM (R=0.997).

To create a calibration curve for correction of turbidity, canine serum samples were spiked with various concentrations of fibrinogen (0-4.5 mM). Assays were performed as described above, and absorbance measurements were taken at both 654 nm and 750 nm. For each sample, the excess net absorbance was calculated by reducing its net absorbance (A₆₅₄-A₇₅₀) by the net absorbance of the unspiked sample. Excess net absorbance was plotted vs. A₇₅₀ for each sample, and the data was fitted to a third order polynomial: Excess net absorbance=1.27×10⁻⁷(A₇₅₀)−3.4617×10⁻⁴(A₇₅₀)+0.65902×(A₇₅₀)−18.71 (R²=0.9991).

Two canine samples exhibiting high turbidity were assayed as described above in duplicate, and absorbance was measured at 654 nm and 750 nm. Using the net absorbance (A₆₅₄-A₇₅₀) with the gadolinium calibration curve yielded average calculated gadolinium concentrations of 12.5 μM and 5.4 μM respectively. However, ICP-MS demonstrated that these samples contained <0.1 μM gadolinium.

For each run, the A₇₅₀ value was converted for turbidity correction using the third order polynomial described above to yield an excess net absorbance value, which was subtracted from the net absorbance to yield an adjusted net absorbance value. The two adjusted net absorbance values for each sample were averaged, and the average was used with the gadolinium calibration curve to yield an uncorrected gadolinium concentration (5.7 μM and −2.0 μM respectively). The hemoglobin concentrations for the samples were 1.34 mg/mL and 0.48 mg/mL respectively; these concentrations were used in conjunction with the hemoglobin calibration curve to yield final corrected gadolinium concentrations of −0.9 μM and −4.3 μM, respectively. Small negative results such as these can be caused by slight interactions between the two interferences, and can be corrected further by using an interaction term in the correction equations. The data underlying these calculations is shown in Table 8.

TABLE 8

| | | | | [Gd] based on | excess | | uncorr. | corr. |
|---|---|---|---|---|---|---|---|---|
| | A₆₅₄ | A₇₅₀ | net A | unadj net A | net A | adj. net A | [Gd] | [Gd] |
| Sample 1, | 2873 | 347 | 2526 | | 174 | 2352 | | |
| Run 1 | | | | | | | | |
| Sample 1, | 2927 | 389 | 2538 | | 193 | 2345 | | |
| Run 2 | | | | | | | | |
| Sample 1, | | | 2532 | 12.5 μM | | 2349 | 5.7 μM | 0.9 μM |
| average | | | | | | | | |
| Sample 2, | 2747 | 406 | 2341 | | 200 | 2141 | | |
| Run 1 | | | | | | | | |
| Sample 2, | 2739 | 394 | 2345 | | 195 | 2150 | | |
| Run 2 | | | | | | | | |
| Sample 2, | | | 2343 | 5.4 μM | | 2145 | −2.0 μM | −4.3 μM |
| average | | | | | | | | |

### Example 15

### Calibration with ICP Standards

Calibration curves were determined using ICP standards (1.633 mM free gadolinium ion in 0.025% nitric acid diluted with water to the desired concentration) and MAGNEVIST® standards (as described above). In the calibration curve of FIG. 13, MAGNEVIST® standards and ICP standards fell on the same line.

Although various specific embodiments of the present invention have been described herein, it is to be understood that the invention is not limited to those precise embodiments and that various changes or modifications can be affected therein by one skilled in the art without departing from the scope and spirit of the invention.

What is claimed is:

1. A method for determining the presence or amount of a gadolinium chelate in a biological sample comprising:
(a) contacting the biological sample with arsenazo III at a pH from about 2 to about 4 or chlorophosphonazo at a pH from about 1 to about 3;
(b) measuring the absorbance of the sample, thereby determining the presence or amount of gadolinium in the sample.

2. The method of claim 1 wherein the dye is arsenazo and the pH is from about 2.0 to about 3.0.

3. The method of claim 1 wherein the dye is chlorophosphonazo and the pH is from about 1.5 to about 2.5.

4. The method of claim 1 wherein the biological sample is not subject to HPLC.

5. The method of claim 1 further comprising inhibiting interference as the result of calcium in the sample.

6. The method of claim 1 further comprising inhibiting interference as the result of ferric ion in the sample.

7. The method of claim 1, wherein the pH is maintained using a glycine, bicine or tricine based buffer.

8. The method of claim 1 wherein the pH is maintained at about 2 to about 4 using a tricine based buffer.

9. The method of claim 8 wherein the tricine based buffer is a tricine-sulfate system.

10. The method of claim 7 wherein the pH is maintained using a triazole based buffer.

11. The method of claim 1, further comprising contacting the sample with an alkylsulfonate having a CMC in the range of 1-100 mM.

12. The method of claim 1, further comprising contacting the sample with a linear C₄-C₉ alkylsulfonate.

13. The method of claim 1 wherein the sample comprises animal plasma or serum.

14. The method of claim 1 wherein the sample is a human sample.

15. The method of claim 1 wherein the sample is contacted with 3-hydroxy-1,2-dimethyl-4(1H)-pyridone.

16. The method of claim 1 wherein the gadolinium chelate comprises gadolinium chelated with DTPA or analogues thereof.

17. A method for determining the presence or amount of a gadolinium chelate in a biological sample, the method comprising:
(a) forming a mixture of a biological sample and a reagent comprising arsenazo III or chlorophosphonazo;
(b) maintaining the pH of the mixture at about 2.0 to about 4.0 when the dye is arsenazo III or at about 1.0 to about 3.0 when the dye is chlorophosphonazo;
(c) measuring the absorbance of the mixture, thereby determining the presence or amount of the gadolinium chelate in the sample.

18. The method of claim 17 wherein the reagent comprises arsenazo III and the pH is from about 2.0 to about 3.0.

19. The method of claim 17 wherein the reagent comprises chlorophosphonazo and the pH is from about 1.5 to about 2.5.

20. The method of claim 17 wherein the biological sample is not subject to HPLC.

21. The method of claim 17 wherein the reagent further comprises 3-hydroxy1,2-dimethyl-4(1H)-pyridone (HDMP).

22. The method of claim 17 wherein the pH is maintained at about 2.0 to about 4.0 using a glycine, bicine or tricine based buffer.

23. The method of claim 22 wherein the buffer is a tricine-sulfate buffer.

24. The method of claim 17 wherein the pH is maintained at about 2.0 to about 4.0 using a triazole based buffer.

25. The method of claim 17 wherein the reagent further comprises an alkylsulfonate having a CMC in the range of 1-100 mM.

26. The method of claim 17 wherein the sample comprises animal plasma or serum.

27. The method of claim 17 wherein the gadolinium chelate comprises gadolinium chelated with DTPA or analogues thereof.

28. A method for determining glomerular filtration (GFR) rate in a mammal comprising:
(a) administering to the mammal an amount of a gadolinium chelate;
(b) determining the concentration level of the chelate in biological samples taken from the animal at an interval or plurality of timepoints following administration of the chelate by contacting the biological samples with arsenazo III at a pH from about 2 to about 4 or chlorophosphonazo at a pH from about 1 to about 3 and measuring the absorbance of the samples;
(c) correlating the concentration levels of the chelate in the samples to GFR of the animal.

29. The method of claim 28 wherein the biological sample is serum or plasma.

30. The method of claim 28 wherein the biological sample is contacted with arsenazo III at a pH from about 2.0 to about 3.0.

31. The method of claim 28 wherein the biological sample is contacted with chlorophosphonazo at a pH from about 1.5 to about 2.5.

32. The method of claim 28 wherein the biological sample is not subject to HPLC.

33. The method of claim 28 further comprising inhibiting calcium interference in the determining of the concentration of the chelate.

34. The method of claim 33 wherein the inhibition of calcium interference comprises contacting the sample with HDMP.

35. The method of claim 28 further comprising inhibiting ferric interference in the determining of the concentration of the chelate.

36. The method of claim 28 wherein the pH is maintained using a glycine, bicine or tricine based buffer.

37. The method of claim 36 wherein the buffer is a tricine-sulfate buffer.

38. The method of claim 28 wherein the pH is maintained using a triazole based buffer.

39. The method of claim 28 wherein the samples are contacted with an alkylsulfonate having a CMC in the range of 1-100 mM when they are contacted with arsenazo III or chlorophosphonazo.

40. The method of claim 28 wherein the sample is a human sample.

41. The method of claim 28 wherein the gadolinium chelate comprises gadolinium chelated with DTPA or analogues thereof.

42. A reagent for use in a colorimetric method for measuring gadolinium chelates in biological samples a dye selected from the group consisting of arsenazo III and chlorophosphonazo, and a buffer for maintaining the reagent at a pH from about 2.0 to about 4.0 when the dye is arsenazo III or at a pH from about 1.0 to about 3.0 when the dye is chlorophosphonazo.

43. The reagent of claim 42, further comprising HDMP.

44. The reagent of claim 42, further comprising an alkylsulfonate having a CMC in the range of 1-100 mM.

45. The reagent of claim 42, wherein the buffer is a glycine-sulfate buffer, a bicine-sulfate buffer, a tricine-sulfate buffer or a triazole-sulfate buffer.

46. A kit comprising the reagent of claim 42 and at least one standard solution of a known gadolinium concentration.

47. A calorimetric method for measuring glomerular filtration rate in an animal comprising:
(a) administering to the animal a gadolinium chelate;
(b) collecting plasma or serum samples from the animal at various times following the administration;
(c) determining the level of gadolinium in the samples by contacting the samples with the reagent of claim 28 and measuring the absorbance of the samples;
(d) comparing the absorbance of the samples to the amount of time following the administration, thereby determining the glomerular filtration rate.

48. The method of claim 47 wherein the sample is not subject to HPLC.

49. The method of claim 47 wherein the gadolinium chelate comprises gadolinium chelated with DTPA or analogues thereof.
